(19) United States
(12) Patent Application Publication
(10) Pub. No.: US 2013/0190525 A1
(43) Pub. Date: Jul. 25, 2013

(54) SYNTHETIC METHODS PERTAINING TO TERT-BUTYL-BENZENE-BASED COMPOUNDS

(71) Applicant: Boston Scientific Scimed, Inc., Maple Grove, MN (US)

(72) Inventors: Joseph Thomas Delaney, JR., Minneapolis, MN (US); Kasyap Seethamraju, Eden Prairie, MN (US); Paul Vincent Grosso, Maple Grove, MN (US)

(73) Assignee: BOSTON SCIENTIFIC SCIMED, INC., Maple Grove, MN (US)

(21) Appl. No.: 13/748,046

(22) Filed: Jan. 23, 2013

Related U.S. Application Data
(60) Provisional application No. 61/589,890, filed on Jan. 24, 2012.

(51) Int. Cl. C07C 41/01; C07C 67/08

(52) U.S. Cl. CPC C07C 41/01; C07C 67/08
USPC 560/98; 568/662

(57) ABSTRACT

According to some aspects, the present disclosure pertains to methods of forming dimethyl 5-tert-butylisophthalate which comprise comprising converting 5-tert-butylisophthalic acid into dimethyl 5-tert-butylisophthalate in synthesis procedures that comprises methanol and a dehydrating agent as chemical reagents. In other aspects, the present disclosure pertains to methods of forming 5-tert-butyl-1,3-bis(1-methoxy-1-methylethyl)benzene that comprise deprotonating 5-tert-butyl-1,3-bis(1-hydroxy-1-methylethyl)benzene with a Brønsted-Lowry superbase and methylating the deprotonated 5-tert-butyl-1,3-bis(1-hydroxy-1-methylethyl)benzene to form the 5-tert-butyl-1,3-bis(1-methoxy-1-methylethyl)benzene.

### STATEMENT OF RELATED APPLICATION

This application claims the benefit of U.S. Ser. No. 61/589,890, filed Jan. 24, 2012 and entitled: “SYNTHETIC METHODS PERTAINING TO TERT-BUTYL-BENZENE-BASED COMPOUNDS,” which is hereby incorporated by reference in its entirety

### BACKGROUND

Thermoplastic elastomers based on difunctional, telechelic soft segments have exceptionally desirable properties. Examples of difunctional telechelic soft segments useful in such thermoplastic elastomers include polyisobutylene-based soft segments, poly(tetramethylene oxide)-based soft segments and pol(ethylene glycol)-based soft segments, among others. A preferred process of making such soft segments containing isobutylene is by carbocationic polymerization involving a difunctional initiator molecule.

There is a whole host of unique and desirable physical and mechanical properties that are offered exclusively by polyisobutylene and polyisobutylene-based materials, including thermal stability, biocompatibility and gas impermeability, among others. These properties can be tuned and further modified in copolymerization strategies with other materials. To form such materials, the carbocationic polymerization of polyisobutylene may be followed by another step, which may or may not be cationic, in which another monomer is polymerized, thereby forming a block copolymer. A difunctional initiator may be used, for example, to synthesize poly(styrene-b-isobutylene-b-styrene) (SIBS) as well as polyurethanes based on a polyisobutylene (PIB) soft segment, among many other copolymers.

Such a polymerization scheme requires a difunctional cationic initiator, an example of which is the di-functional living cationic polymerization initiator,

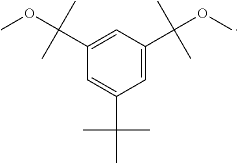

This compound (CAS#108180-34-3) is known as 1-(1,1-dimethylethyl)-3,5-bis(1-methoxy-1-methylethyl)-benzene, or alternatively as 1,3-bis(2-methoxy-2-propyl)-5-tert-butylbenzene, 1,3-bis(1-methoxy-1-methylethyl)5-tert-butylbenzene, or 5-tert-butyl-1,3-bis(1-methoxy-1-methylethyl)benzene. This compound is referred to herein as “hindered dicumyl ether” or HDCE.

A related compound that has also been used as a difunctional initiator for living cationic polymerization is

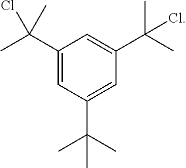 [SMILES: CC(C)(C)c1cc(C(C)(C)Cl)cc(C(C)(C)Cl)c1]

This compound (CAS#89700-89-0) is known as 1,3-bis(1-chloro-1-methylethyl)-5-(1-dimethylethyl)benzene or alternatively as 1,3-bis(1-chloro-1-methylethyl)-5-tert-butylbenzene. This compound is referred to herein as “hindered dicumyl chloride” or HDCC.

Due to the high cost of materials resulting from the need for difunctional initiators such as HDCE and HDCC, which are specialty chemicals, the use of cationically polymerized telechelic, difunctional soft segments, including telechelic, difunctional polyisobutylene soft segments, is currently limited to specialized, high-value-added applications, for instance, drug delivery coatings for stents. However, if the cost of the initiator can be brought down closer to commodity levels, a wide range of applications will become economically viable.

### SUMMARY OF THE INVENTION

According to some aspects, the present disclosure pertains to methods of forming dimethyl 5-tert-butylisophthalate which comprise converting 5-tert-butylisophthalic acid into dimethyl 5-tert-butylisophthalate in synthesis procedures that comprise methanol and a dehydrating agent as chemical reagents.

In other aspects, the present disclosure pertains to methods of forming 5-tert-butyl-1,3-bis(1-methoxy-1-methylethyl)benzene that comprise deprotonating 5-tert-butyl-1,3-bis(1-hydroxy-1-methylethyl)benzene with a Brønsted-Lowry superbase and methylating the deprotonated 5-tert-butyl-1,3-bis(1-hydroxy-1-methylethyl)benzene to form the 5-tert-butyl-1,3-bis(1-methoxy-1-methylethyl)benzene.

These and other aspects, embodiments and advantages of the present invention will become immediately apparent to those of ordinary skill in the art upon review of the Detailed Description and claims to follow.

### DETAILED DESCRIPTION

A more complete understanding of the present disclosure is available by reference to the following detailed description of numerous aspects and embodiments. The detailed description which follows is intended to illustrate but not limit the invention.

HDCE may be formed in three process steps, which are depicted in the following scheme:

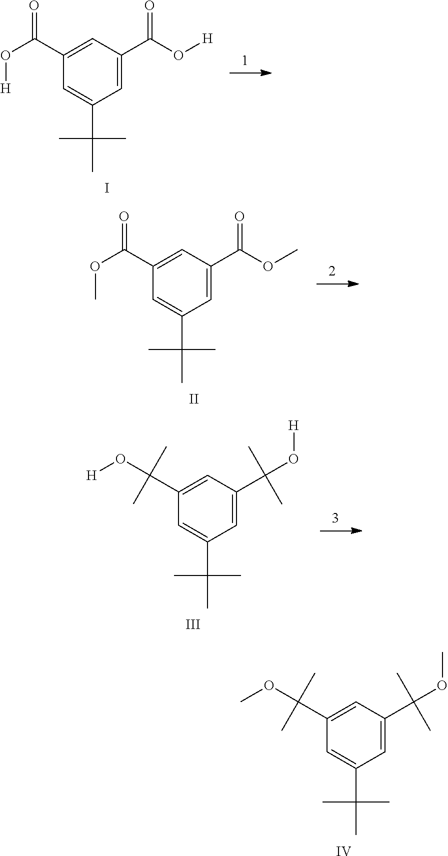

As outlined in B. Wang et al., Polymer Bulletin (Berlin, Germany), 1987, 17, 205-21, the above method steps are as follows: Step 1. Fischer-Speier esterification of 5-tert-butylisophthalic acid (Formula I to produce dimethyl 5-tart-butylisophthalate (Formula II). Step 2. Grignard reaction of dimethyl 5-tert-butylisophthalate (Formula II) with methylmagnesium bromide to produce 1-(1,1-dimethylethyl)-3,5-bis(1-hydroxy-1-methylethyl)benzene, also referred to as 1,3-bis(2-hydroxy-2-propyl)-5-tert-butylbenzene, 5-tert-butyl-1,3-bis(1-hydroxy-1-methylethyl)benzene or 1,3-bis(1-hydroxy-1-methylethyl)-5-tert-butylbenzene (Formula III). This compound is referred to herein as “hindered dicumyl alcohol” or HDCA. Step 3. Williamson ether synthesis of HDCA (Formula III) with methanol catalyzed by sulfuric acid under reflux conditions to yield HDCE (Formula IV.

The present disclosure addresses drawbacks associated with the first and third method steps in the above synthesis scheme.

The dimethyl 5-tert-butylisophthalate product of the first (initial) step is also contemplated as a starting material in the synthesis of HDCC. In this regard, the improvements detailed below for the first (initial) step in the synthesis of HDCE are also applicable to the synthesis of HDCC.

Initial Step As noted above, it is presently known to use Fischer-Speier esterification of 5-tert-butylisophthalic acid (Formula I) to produce dimethyl 5-tert-butylisophthalate (Formula I) in the following process step:

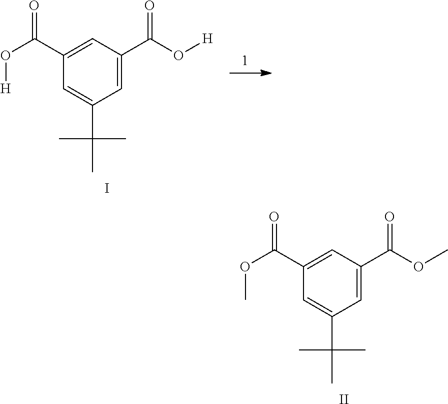

There are inefficiencies in the reaction currently performed in which the diacid starting material is combined with an enormous excess of methanol in the presence of sulfuric acid catalyst over 14 to 18 hours. For instance, in Comparative Example 1 of the present disclosure, 200 mL (158 grams, 4.94 moles) of methanol is used to esterify 10 grams (0.045 moles) of 5-tert-butylisophthalic acid, which constitutes a 110-fold molar excess. The yield of dimethyl 5-tert-butylisophthalate was 8.14 grams, 72% of theoretical. Thus, the reaction has a yield that would benefit from improvement, and the reaction requires larger scale equipment due to the enormous excess of methanol.

In accordance with one embodiment, a procedure is provided wherein a dehydrating agent is employed during diesterification to provide reaction conditions for the diesterification step that allow a reduced excess of methanol and provide for enhanced yield. For instance, in Example 1 below, molecular sieves are used as dehydrating agents for the reaction of 5-tert-butylisophthalic acid (25.0 grams, 0.112 moles) with a 27-fold molar excess anhydrous methanol (125 mL, 99 grams, 3.00 moles) in the presence of an acid catalyst (e.g., 96-98% sulfuric acid catalyst; 1.50 mL, 2.7 grams) to achieve a yield of 27.43 grams, or 98% of theoretical.

Dehydrating agents other than molecular sieves that may be used include silica gels, alumina, calcium hydride, and calcium oxide, among other dehydrating agents.

Acid catalysts other than sulfuric acid that may be used include p-toluenesulfonic acid, trifluoroacetic acid and triflic acid, among others.

In other embodiments, dehydrating agents are employed that react irreversibly with any water present during the diesterification to provide reaction conditions for the diesterification step that require a smaller excess of methanol than the present method, thus allowing the same amount of product diester to be made in smaller equipment or allowing a greater amount of product diester to be made in existing equipment.

In these embodiments, 5-tert-butylisophthalic acid, a chemical dehydrating agent (e.g., a phosphorous dehydrating agent such as phosphorous oxychloride or phosphorous pentoxide, among others), methanol, an optional solvent (e.g., dichloromethane, etc.) and an optional base (e.g., pyridine, etc.) are combined to produce dimethyl 5-tert-butylisophthalate.

For instance, in one specific embodiment, phosphorus oxychloride (0.5 mL, 5.5 mmol) is added at room temperature to a solution of 5-tert-butylisophthalic acid (1.1 g, 5 mmol), and pyridine (0.4 mL, 5 mmol) in dichloromethane (25 mL). The mixture is stirred at room temperature for 15 min. Then, methanol (0.26 g, 8 mmol) and pyridine (1.2 mL, 15 mmol) are added at 5° C. The resulting solution is stirred at room temperature for 3 h. The mixture is washed with water (15 mL), followed by 0.1 N hydrochloric acid (10 mL), and then again with water (15 mL); the organic layer is separated and dried over sodium sulfate. In this procedure, only a small (e.g., 1.6-fold) excess of methanol is used for esterification.

Alternate phosphorus dehydrating agents other than phosphorous oxychloride include phenyldichlorophosphate, phenyl N-phenylphosphoramidochloridate, phosphorous pentachloride, and N,N′-bis(2-oxo-3-oxazolidinyl)phosphorodiamidic chloride, among others.

Other examples of dehydrating agents include cyanuric chloride, acyloxisilanes, polymer-bound oxazolines, dicyclohexylcarbodiimide, 4-(NIN-dimethylamino) pyridine, 1-fluoro-2,4,6-trinitrobenzene/4-(N,N-dimethylamino)pyridine, chloroformates, trimethyl orthoformate, acylphosphonates, dialkylsulphites, orthosilicates such as tetramethoxysilane and trimethoxy methysilane, and sulfonyl chlorides, among others.

### Middle Step

A beneficial middle step is the Grignard reaction of dimethyl 5-tert-butylisophthalate with methylmagnesium bromide to produce HDCA. See B. Wang et al., Polymer Bulletin (Berlin, Germany), 1987, 17, 205-21.

### Final Step

As noted above, it is presently known to react HDCA (Formula III) with methanol catalyzed by sulfuric acid under reflux conditions to yield HDCE (Formula IV):

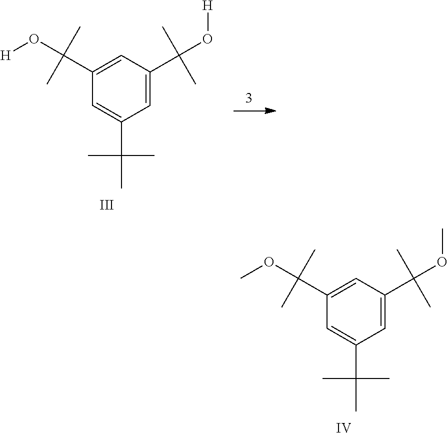

While the reported value for this last step is 80% in the literature, it has been found that, in practice, this value is significantly lower. The reaction conditions used (refluxing with methanol in concentrated sulfuric acid) are conducive to a number of competing side-reactions. In this regard, sulfuric acid catalysis and heat are reasonably good reaction conditions to drive E2 elimination, resulting in the dehydration of the alcohol starting material, where water is driven off, yielding an olefin functional group. There is also the possibility of a second side reaction, i.e., β-elimination of methanol from the HDCE, where a methoxy group of the finished product is driven off to yield an olefin functional group, destroying an already-formed product during the process. Importantly, difunctionality of the HDCE product is critical to its utility as a polymerization initiator. Consequently, a side product with an olefin functional group instead of two methoxy groups is an unwanted impurity in HDCE, and its occurrence should be minimized.

Other unwanted side reactions may take place in addition to those discussed above, including polycondensation reactions and addition reactions to olefins.

One result of the preceding side reactions is that, after the final reaction step is complete, the crude product requires extensive recrystallization as part of the work-up. Because this process is laborious, it is an additional cause for loss of product. For instance, in Comparative Example 2 below, the yield of recrystallized product was only 30% of theoretical. The fact that this is the last step in the synthetic route makes the yield loss that much more costly. Thus, while the cost of the reagents is quite low, low yields and byproducts make this reaction step an unattractive technique.

On contrast, the present disclosure employs methylating techniques for tertiary alcohols that offer reduced risks of significant side reactions. In these techniques, strong bases, preferably, Brønsted-Lowry superbases, are employed to deprotonate the tertiary alcohols, converting them into strong nucleophiles which are reacted with electrophilic methylating reagents.

Superbases with anions that form gaseous products when protonated ensure that the reaction is not only highly favored, but also irreversible, are preferred in some embodiments. In these embodiments, the kinetic barrier for the reaction is much lower, making the reaction more favorable at lower temperatures, typically in the range of −78° C. to ambient temperature. By using lower temperatures and dispensing with the non-selective catalyst of concentrated sulfuric acid, numerous side-reactions can be minimized.

For example, in one beneficial embodiment, a solution of HDCA in solvent (e.g., THF, etc.) is added to a superbase (e.g., NaH, etc.) over a period of several minutes. The resulting mixture is stirred until hydrogen generation is complete at which point methylating agent (e.g., methyl iodide, etc.) is added. The reaction mixture is stirred for a suitable time (e.g. several hours) to complete the reaction. In Example 2 below, a technique of this type produced a yield that was 93% of theoretical with high product purity. Without wishing to be bound by theory, the overall reaction may be illustrated schematically as follows:

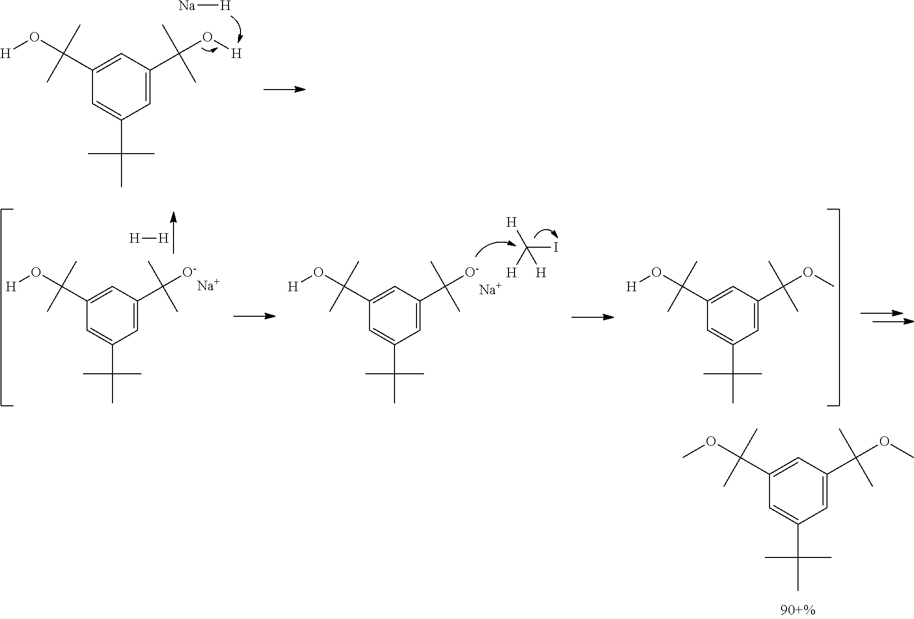

Alternative inorganic and organometallic Brønsted-Lowry superbases beyond sodium hydride include additional metal hydrides such as potassium hydride, lithium hydride, sodium amide, lithium nitride, and organolithium salts including alkyl lithium compounds such as methyl lithium and isomers of but lithium, lithium amides such as lithium diisopropylamide, lithium diethylamide and lithium bis(trimethylsilyl)amide, and a combination of n-butyllithium and potassium tert-butoxide, among others. Without wishing to be bound by theory, preferred Brønsted-Lowry bases include those where the pK_{A} of the conjugate is as high as possible, such that the conjugate is more likely to seize a proton and retain it. The aromatic tertiary alcohol intermediate in the present scheme (HDCA) has a pKa of approximately 17. Consequently, a strong base is preferred where the conjugate acid's pKa is significantly higher than 17, preferably at least 2 units higher for deprotonation to go effectively to completion.

Alternative methylating reagents beyond methyl iodide include other methyl halides such as methyl bromide, as well as additional methyl compounds such as dimethyl carbonate, dimethyl sulfate, methyl 4-toluenesulfonate, methyl fluorosulfonate, methyl methanesulfonate, methyl trifluoromethanesulfonate, tetramethyl orthosilicate, tetramethylammonium chloride (as well as other methylated quaternary ammonium salts), trimethoxy methyl silane, trimethyl borate, trimethyl orthoformate (as well as other trimethyl ortho esters of organic acids), and trimethyl phosphate, among others.

Alternative solvents beyond THF include ethyl ether and dioxane, among others.

Several examples will now be provided which illustrate, but do not limit, the present disclosure. Unless indicated otherwise, all reagents were obtained from Sigma-Aldrich.

### Example 1

### Dimethyl 5-tert-butylisophthalate Prepared Using Molecular Sieves

5-tert-Butylisophthalic acid (25.0 grams, 0.112 moles) was placed in a 500-mL, three-neck, round-bottomed flask along with a magnetic stir bar. The necks of the flask were fitted with a thermocouple, a septum and the body of a Soxhlet extractor. A flow of dry nitrogen was introduced to the flask via a needle that pierced the septum. 30 grams of 3 A molecular sieves, which had been dried overnight at 150° C. under a nitrogen atmosphere, were loaded into a 25 mm×90 mm extraction thimble. The thimble was inserted into the extractor body and a condenser was placed atop the body. A nitrogen outlet, connected to a bubbler, was attached to the top of the condenser.

Anhydrous methanol (125 mL, 99 grams, 3.00 moles; a 27-fold molar excess versus 5-tert-butylisophthalic acid) was added via cannula to the round-bottom flask. Sulfuric acid catalyst (96-98%; 3.75 mL, 6.9 grams) was added next. The mixture was heated to reflux temperature (65° C.). The cloudy, white mixture became clear as the reaction proceeded. Reflux continued for 30 hours.

Upon cooling to 45° C. after reflux, the clear solution became a dense mass of white crystals, wet with methanol. The crystals were collected on a sintered-glass funnel and washed twice with 50-mL portions of methanol that had been cooled to −20° C. The product was allowed to dry in the funnel. The yield was 27.43 grams, 98% of theoretical.

FTIR analysis of the product showed a very strong ester carbonyl peak at 1718 cm⁻¹ and sharp absorptions of medium intensity at 2970 cm⁻¹ due to C—CH₃ and at 2870 cm⁻¹ in the C—H stretching region. This contrasts with the starting material, which showed a very strong carboxylic acid dimer peak at 1690 cm⁻¹ and a broad, diffuse peak in the region between 2500 and 3250 cm⁻¹. The proton NMR spectrum was consistent with the structure of dimethyl 5-tert-butylisophthalic acid, with protons due to the methyl esters visible at 3.95 ppm. An NMR-based analysis showed a purity of 91.5%, with the impurity being unreacted acid.

### Comparative Example 1

### Dimethyl 5-tert-butylisophthalic Acid Prepared Using a Large Molar Excess of Methanol

5-tert-Butylisophthalic acid (10.0 grams, 0.045 moles) was placed in a 500-mL, three-neck, round-bottomed flask along with a magnetic stir bar. The necks of the flask were fitted with a thermocouple, a nitrogen inlet and a condenser. A flow of dry nitrogen was introduced to the flask. A nitrogen outlet, connected to a bubbler, was attached to the top of the condenser. Anhydrous methanol (200 mL, 158 grams, 4.94 moles; a 110-fold molar excess versus 5-tert-butylisophthalic acid) was added to the round-bottom flask. Sulfuric acid catalyst (96-98%; 1.50 mL, 2.7 grams) was added next. The mixture was heated to reflux temperature (65° C.). The cloudy, white mixture became clear as the reaction proceeded. Reflux was maintained for 24 hours.

Upon cooling the reaction, a small quantity of fine, white crystals appeared. The flask was cooled to near 0° C. and the white product was collected on a sintered-glass funnel. The solid was washed with a few mL of cold methanol and dried in the fritted filter. The yield of dimethyl 5-tert-butylisophthalic acid was 8.14 grams, 72% of theoretical.

### Example 2

### Preparation of 5-tert-butyl-1,3-bis(1-methoxy-1-methylethyl)benzene (HDCE) Via Etherification with Methanol in the Presence of a Superbase

All glassware used in this example was oven-dried overnight and assembled hot and/or under a stream of dry nitrogen. The starting material, 5-tert-butyl-1,3-bis(1-hydroxy-1-methylethyl)benzene (HDCA) may be formed by the Grignard reaction of dimethyl 5-tert-butylisophthalate (see Example 1) with methylmagnesium bromide to produce HDCA, as is known in the art. See B. Wang et al., Polymer Bulletin (Berlin, Germany), 1987, 17, 205-21.

In a 500-mL boiling flask, HDCA (10.0 g, 0.0399 moles), was dissolved in 200 mL anhydrous THF. The THF solution was transferred via cannula to a 250-mL pressure-equalizing addition funnel. The funnel and a nitrogen inlet were fitted to a Claisen adapter and the adapter placed on one neck of a 500-mL, three-necked, round-bottom flask. A thermocouple and a nitrogen outlet, connected to a bubbler, were inserted in the remaining necks of the flask. Sodium hydride (5.26 g of a 60% dispersion in mineral oil; equivalent to 3.16 g or 0.132 moles NaH) was added to the flask and washed with five 25-mL portions of anhydrous methylcyclohexane to remove mineral oil.

The THF solution was added over 20 minutes to the flask with magnetic stirring. The temperature of the white slurry in the flask was maintained at around 20-25° C. Hydrogen generation was complete after 60 minutes. Methyl iodide (12.07 g, 5.29 mL, 0.085 moles) was next added to the flask via syringe over 30 seconds. Stirring continued at room temperature for 24 hours. 150 mL of methylene chloride was added to the flask, then excess sodium hydride was consumed by addition of isopropanol. The reaction mixture was diluted with 200 mL ethyl ether, and the solution was extracted with saturated aqueous sodium chloride. The combined aqueous fractions were extracted in turn with two 75-mL portions of ether. All the organic fractions were combined and dried over sodium sulfate. Removal of the solvents on a rotary evaporator yielded 10.35 g (93% of theoretical) of an amber oil that slowly crystallized in the cold.

The proton NMR spectrum was consistent with the structure of 5-tert-butyl-1,3-bis(1-methoxy-1-methylethyl)benzene, with protons due to the methyl ethers visible at 3.21 ppm. An NMR-based analysis showed a purity of 99.9% with no detectable olefin impurity.

### Comparative Example 2

### Preparation of 5-tert-butyl-1,3-bis(1-methoxy-1-methylethyl) benzene (HDCE) Via Etherification with Methanol in the Presence of a Strong Acid Catalyst

A two-liter flask was equipped with a reflux condenser and magnetic stir bar. The flask was charged with 5-tert-butyl-1,3-bis(1-hydroxy-1-methylethyl)benzene (HDCA) (180.0 g, 0.719 moles) and methanol (280 mL). The mixture was stirred to effect dissolution and a solution of 0.072 mL of concentrated sulfuric acid in 300 mL of methanol was added. The solution was stirred at reflux for 6 hours. The cooled solution was extracted with three 420-mL portions of hexane, and the combined hexane phases were washed with 1.3 L of water. The organic phase was dried over 75 g anhydrous sodium sulfate, and hexane was removed on a rotary evaporator. Recrystallization of the product from hexane multiple times, until less than 2% olefinic impurity remained, yielded 60 g of HDCE (30% of theoretical).

Although various embodiments are specifically illustrated and described herein, it will be appreciated that modifications and variations of the present invention are covered by the above teachings and are within the purview of any appended claims without departing from the spirit and intended scope of the invention.

1. A method of forming 5-tert-butyl-1,3-bis(1-methoxy-1-methylethyl)benzene (Formula IV) comprising deprotonating 5-tert-butyl-1,3-bis(1-hydroxy-1-methylethyl)benzene (Formula III) with a Brønsted-Lowry superbase and methylating the resulting deprotonated 5-tert-butyl-1,3-bis(1-hydroxy-1-methylethyl)benzene by reacting said deprotonated 5-tert-butyl-1,3-bis(1-hydroxy-1-methylethyl)benzene with a methylating agent to form said 5-tert-butyl-1,3-bis(1-methoxy-1-methylethyl)benzene.

2. The method of claim 1 wherein the Brønsted-Lowry superbase results in hydrogen gas as a byproduct of said deprotonation process.

3. The method of claim 1 wherein the Brønsted-Lowry superbase is a metal hydride.

4. The method of claim 1 wherein the Brønsted-Lowry superbase is selected from sodium hydride, potassium hydride, sodium amide and lithium nitride.

5. The method of claim 1 wherein the Brønsted-Lowry superbase comprises an organolithium salt.

6. The method of claim 1 wherein the methylating agent is a methyl halide.

7. The method of claim 1 wherein the methylating agent is methyl iodide.

8. The method of claim 1 wherein the methylating agent is selected from dimethyl carbonate, dimethyl sulfate, methyl 4-toluenesulfonate, methyl bromide, methyl fluorosulfonate, methyl methanesulfonate, methyl trifluoromethanesulfonate, tetramethyl orthosilicate, tetramethylammonium chloride, trimethoxy methyl silane, trimethyl borate, trimethyl orthoformate and trimethyl phosphate.

9. The method of claim 1 wherein the deprotonating and methylating processes are performed in a solvent, and wherein the solvent comprises tetrahydrofuran.

10. The method of claim 1, wherein the yield is at least 90% of theoretical with a product purity of at least 95%.

11. A method of forming dimethyl 5-tert-butylisophthalate (Formula II) comprising converting 5-tert-butylisophthalic acid (Formula I) into dimethyl 5-tert-butylisophthalate by reacting the 5-tert-butylisophthalic acid and methanol in the presence of an acid catalyst while employing a dehydration agent.

12. The method of claim 11, wherein the acid catalyst comprises sulfuric acid.

13. The method of claim 11, wherein the dehydration agent is a solid-phase dehydration agent.

14. The method of claim 13, wherein the solid phase dehydration agent is selected from molecular sieves, silica gel, alumina, calcium hydride, and calcium oxide.

15. A method of forming dimethyl 5-tert-butylisophthalate (Formula II) comprising converting 5-tert-butylisophthalic acid (Formula I) into dimethyl 5-tert-butylisophthalate in a synthesis procedure comprising the 5-tert-butylisophthalic acid, methanol, a chemical dehydration agent, an optional solvent and an optional base as chemical reagents.

16. The method of claim 15, wherein the chemical dehydrating agent is a phosphorus dehydrating agent selected from phosphorous oxychloride, phenyldichlorophosphate, diphenylchlorophosphate, phenyl N-phenylphosphoramidochloridate, and N,N′-bis(2-oxo-3-oxazolidinyl)phosphorodiamidic chloride.

17. The method of claim 15, wherein the chemical dehydrating agent is selected from cyanuric chloride, acyloxisilanes, polymer-bound oxazolines, dicyclohexylcarbodiimide, 4-(NIN-dimethylamino)pyridine, 1-fluoro-2,4,6-trinitrobenzene/4-(N,N-dimethylamino) pyridine, chloroformates, acylphosphonates, dialkylsulphites, and sulfonyl chlorides.

18. The method of claim 15, wherein the dehydrating agent comprises phosphorous oxychloride.

19. The method of claim 15, comprising said base as a chemical reagent, wherein said base comprises pyridine.

20. The method of claim 15, comprising said solvent as a chemical reagent, wherein said solvent comprises tetrahydrofuran.
